(19) United States
(12) Patent Application Publication
(10) Pub. No.: US 2021/0145655 A1
(43) Pub. Date: May 20, 2021

(54) SURGICAL PAD AND METHOD OF OBTAINING THE SAME

(71) Applicant: YEDITEPE UNIVERSITESI, Istanbul (TR)

(72) Inventors: Mahmut Gazi YASARGIL, Istanbul (TR); Dianne Cathryn Helena YASARGIL, Istanbul (TR); Basar ATALAY, Istanbul (TR)

(73) Assignee: YEDITEPE UNIVERSITESI, Istanbul (TR)

(21) Appl. No.: 16/622,305

(22) PCT Filed: Feb. 5, 2018
(86) PCT No.: PCT/TR2018/050040

§ 371 Date: Dec. 13, 2019

(51) Int. Cl. A61F 13/44

(52) U.S. Cl. CPC A61F 13/44

(57) ABSTRACT

A surgical pad having a head part including an intermediate layer including an X-ray detectable material for use in surgical operations, particularly in brain and spinal cord operations. In the surgical pad, the intermediate layer is provided between layers, which are produced from 100% cotton and have a geometric shape that is manually formed.

### CROSS REFERENCE TO THE RELATED APPLICATION

This application is the national phase entry of International Application No. PCT/TR2018/050040 filed on Feb. 5, 2018, the entire contents of which are incorporated herein by reference.

### TECHNICAL FIELD

The present invention relates to surgical pads which are particularly used in microsurgical operations such as brain and spinal cord operations.

### BACKGROUND

As is known, in surgical operations, particularly in brain and spinal cord surgical operations, pads are used to absorb bodily fluids such as blood, lymph, and cerebrospinal fluid. In addition to the requirement that the pads used in these operations should be small in size, they should also reflect X-rays, i.e. should be radiopaque, in order to be detected radiologically during the surgery. Furthermore, there should not be any sticking on the parts of the pads contacting the tissue.

The state of the art patent application no. U.S. Pat. No. 3,911,922 discloses a surgical sponge which is made of a porous fabric coated on both sides with a flexible foamed polymer, both surfaces of which can absorb liquids. The sponge comprises an X-ray detectable material.

Another patent application no. EP2641560A discloses a surgical pad, which is coated with a resin that prevents any residues from the pad surface to remain on the site during an operation, is comprised of a plurality of absorbent fabric structure layered on top of one another, and has a layer containing X-ray radiopaque substance.

Another patent application no. US2005109347 discloses a surgical towel comprising an X-ray detectable material. The surgical towel is made of a sheet of woven fabric having an X-ray detectable material. The X-ray detectable material is sewn or otherwise attached to the fabric. The hem is stitched with a thread of a different color to show that the towel comprises an X-ray detectable material.

### SUMMARY

The object of the present invention is to provide a surgical pad, which is suitable in size for use in microsurgical operations such as brain and spinal cord surgical operations and which does not stick to the body tissue, and a surgical pad obtaining method.

In order to achieve the said object, the invention is a surgical pad having a head part including an intermediate layer comprising an X-ray detectable material for use in surgical operations, particularly in brain and spinal cord operations. In the surgical pad, the intermediate layer is provided between layers, which are produced from 100% cotton and have a geometric shape that is manually formed. Thus, a radiopaque surgical pad, which is of desired small sizes for microsurgical operations, and which does not stick to the body tissue, is provided.

Ina possible embodiment of the invention, the head part is produced from a pressed cotton or a hydrophilic cotton material. This way, a head part that does not stick to the body tissue is obtained.

In another possible embodiment of the invention, the head part has a square, rectangular or circular geometric shape. Thus, surgical pads of different geometric shapes are provided for microsurgical operations.

Another possible embodiment of the invention comprises a tail part connected to the head part. Thus, a tail part is provided for the surgical pads.

In another possible embodiment of the invention, the tail part is stitched to the head part such that it is marked with an X-ray detectable material. Thus, the surgical pad is marked with a radiopaque stitch.

In another possible embodiment of the invention, the tail part is a thread produced from a cotton material. This way, the tail part is produced from a material that does not stick to the body tissue.

In another possible embodiment of the invention, a surgical pad bunch is formed by connecting the tail parts of a plurality of surgical pads. Thus, a surgical pad bunch comprising a plurality of surgical pads connected to each other is obtained, for packaging.

In a possible embodiment of the invention, the X-ray detectable material is an indicator comprising barium oxide or ion.

In order to achieve the said object, the invention is a method of obtaining surgical pad having an angular geometry for use in surgical operations, particularly brain and spinal cord operations. The method of obtaining a surgical pad comprises the steps of manually folding a pressed cotton (milk filter) along the length thereof to form an upper layer and a lower layer and cutting them to predetermined lengths, placing an X-ray detectable material between the upper layer and the lower layer to form a radiopaque intermediate layer, cutting at least one edge part of the pressed cotton to acquire the final form and obtaining the head part, stitching a cotton thread to the head part such that it is marked with an X-ray detectable material and thus forming a tail part. Thus, a method of manually obtaining surgical pads is introduced.

In an alternative embodiment of the invention, the lower and upper layers at the head part of the surgical pad can be a milk filter produced from cotton material. In another alternative embodiment of the invention, the surgical pad can be obtained in different geometric shapes which are angular or not.

In another alternative embodiment of the invention, the surgical pad bunch is obtained by connecting the tail parts of a plurality of surgical pads for packaging.

### BRIEF DESCRIPTION OF THE DRAWINGS

FIG. 1 shows a representative view of the surgical pad having a circular head part.

FIG. 2 shows a representative view of the surgical pad bunch comprising surgical pads having circular head parts for packaging.

FIG. 3 shows a representative view of the surgical pad having a rectangular head part.

FIG. 4 shows a representative view of the surgical pad bunch comprising surgical pads having rectangular head parts for packaging.

FIG. 5 shows a representative view showing the layers forming the head part of the surgical pad.

### REFERENCE NUMBERS

10. Surgical pad
11. Head part
111. Lower layer
112. Upper layer
113. Intermediate layer
12. Tail part
13. Stitching part
14. Connection point
15. Edge part
16. End part
20. Surgical pad bunch

### DETAILED DESCRIPTION OF THE EMBODIMENTS

In this detailed description, a surgical pad (10) and a method for obtaining the said surgical pad (10) are described by examples that are only provided to facilitate better understanding of the invention without producing any limiting effect.

The surgical pad (10), which is used for absorbing the bodily fluids in microsurgical operations such as brain and spinal cord operations, is basically comprised of a head part (11) an a tail part (12). The head part (11) is produced from a material that will enable the surgical pads (10) to absorb bodily fluids. An end of the tail part (12) is connected to the head part (11) by stitching from a stitching part (13). The other end of the tail part (12) is free.

Referring to FIG. 5, the head part (11) includes a lower layer (111), and an upper layer (112) corresponding to the lower layer (111). The upper layer (112) and the lower layer (111) are produced from 100% cotton and have a geometric shape that is manually formed. In more detail, the said % 100 cotton material is pressed cotton or hydrophilic cotton. In a possible embodiment, the head part (11) may have a square, rectangular or circular shape. An intermediate layer (113) comprising an X-ray detectable material is provided between the said lower layer (111) and upper layer (112). In a possible embodiment, the X-ray detectable material is an indicator comprising barium oxide or ion.

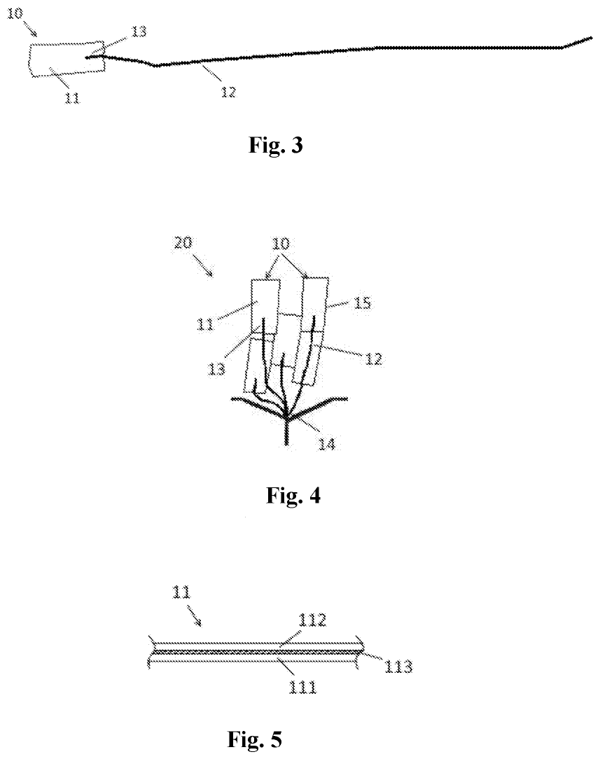

The surgical pad (10) having a rectangular head part (11) given in FIG. 3 is obtained by means of the following method. First of all, the pressed cotton is manually folded along the length thereof so as to produce the upper layer (112) and the lower layer (111). Then the pressed cotton is cut from the folded parts to predetermined lengths by the help of a cutter. Thus the lower layer (111) and the upper layer (112) are provided such that they have matching geometries. An intermediate layer (113) is formed between the said upper and lower layers (111, 112) by placing an X-ray detectable material. At least one edge part (15) of the pressed cotton is cut to acquire the final form and thus the head part (11) is obtained. A cotton thread is stitched to the head part (11) such that it is marked with an X-ray detectable material and this way, the tail part (12) and the head part (11) are connected to each other. Thus a rectangular surgical pad (10) is obtained. As the final procedure, referring to FIG. 4, a plurality of surgical pads (10) are connected through at least one connection point (14) thereby providing a surgical pad bunch (20). The said surgical pad bunch (20) has a similar appearance with a grape bunch. The head parts (11) of the surgical pads (10) in the surgical pad bunch (20) at least partially contact each other and are positioned so as to neighbor each other. In a possible embodiment, the surgical pad bunch (20) is provided by connecting at least five surgical pads (10) to each other. The said head part (10) can have any angular geometrical shape such square and rectangle.

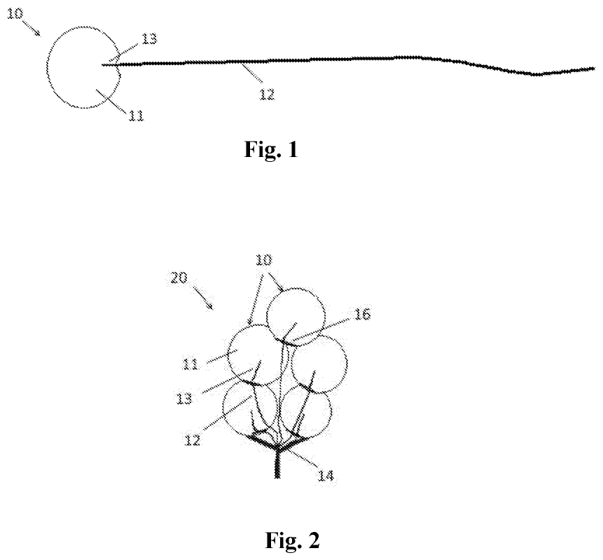

The surgical pad (10) having a circular head part (11) given in FIG. 1 is obtained by means of the following method. First of all, the hydrophilic cotton is manually formed into a geometric shape at desired pad sizes. An X-ray detectable material is placed on the hydrophilic cotton to form an intermediate layer (113). The said hydrophilic cotton will form the lower layer (111) and the upper layer (112) of the head part (11). When the hydrophilic cotton is wrapped around the X-ray detectable material, the X-ray detectable material is positioned between the upper layer (112) and the lower layer (111). Then, the lower layer (111) and the upper layer (112) are connected to the each other when an end part (16) of the head part (11) which is open is stitched. This way, the head part (11) is formed into a disc having an upper layer (112) and lower layer (111). The said end part (16) is provided in at least one part of the periphery of the hydrophilic cotton. The head part (11) is obtained when the portions exceeding the periphery of the hydrophilic cotton are cut such that the hydrophilic cotton will acquire its final form. A cotton thread is stitched to the head part (11) such that it is marked with an X-ray detectable material and thus the tail part (12) and the head part (11) of the surgical pad (10) are connected to each other. Thus a circular surgical pad (10) is obtained. As the final procedure, referring to FIG. 2, a plurality of surgical pads (10) are connected through at least one connection point (14) thereby providing a surgical pad bunch (20). The said surgical pad bunch (20) has a similar appearance with a grape bunch. The head parts (11) of the surgical pads (10) in the surgical pad bunch (20) at least partially contact each other and are positioned so as to neighbor each other. In a possible embodiment, the surgical pad bunch (20) is provided by connecting at least five surgical pads (10) to each other. The said head part (11) can have any geometric shape without corners such as a circle. The surgical pad (10) and the surgical pad bunch (20) obtained by means of any of the above mentioned methods are sterilized thereby obtaining surgical pads (10) that are suitable to be used in surgical operations.

What is claimed is:

1. A surgical pad, comprising: a head part including an intermediate layer; an X-ray detectable material for use in surgical operations including brain and spinal cord operations, the intermediate layer is provided between side layers wherein the side layers are produced from 100% cotton and have a geometric shape, wherein the geometric shape is manually formed at desired sizes; a tail part is connected to the head part.

2. The surgical pad according to claim 1, wherein the head part is produced from a pressed cotton or a hydrophilic cotton material.

3. The surgical pad according to claim 1, wherein the head part has a square geometric shape, a rectangular geometric shape or a circular geometric shape.

4. (canceled)

5. (canceled)

6. The surgical pad according to claim 1, wherein the tail part is a thread produced from the pressed cotton or the hydrophilic cotton material.

7. (canceled)

8. The surgical pad according to claim 1, wherein the X-ray detectable material is an indicator comprising barium oxide or barium oxide ion.

9. A method of obtaining surgical pad having an angular geometry for use in surgical operations including brain and spinal cord operations, wherein the method of obtaining surgical pad comprises the steps of
manually folding a pressed cotton along a length thereof to form an upper layer and a lower layer and cutting the upper layer and the lower layer to predetermined lengths,
placing an X-ray detectable material between the upper layer and the lower layer to form a radiopaque intermediate layer,
cutting at least one edge part of the pressed cotton to acquire a final form and obtaining a head part,
stitching a cotton thread to the head part, wherein the head part is marked with the X-ray detectable material forming a tail part.

10. A method of obtaining surgical pad having a non-angular geometry for use in surgical operations including brain and spinal cord operations, wherein the method of obtaining surgical pad comprises the steps of
manually forming a hydrophilic cotton into a geometric shape at desired pad sizes, placing an X-ray detectable material into the hydrophilic cotton to form a radiopaque intermediate layer,
wrapping the hydrophilic cotton around the X-ray detectable material, stitching an end part, and forming into a disc having an upper layer and a lower layer,
obtaining the head part having the non-angular geometry by cutting portions exceeding a periphery of the hydrophilic cotton, wherein the hydrophilic cotton acquires a final form,
stitching a cotton thread to the head part, wherein the head part is marked with the X-ray detectable material forming a tail part.

11. (canceled)

12. The surgical pad according to claim 8, wherein the head part is produced from the pressed cotton or the hydrophilic cotton material.

13. The surgical pad according to claim 8, wherein the head part has the square geometric shape, the rectangular geometric shape or the circular geometric shape.

14. The surgical pad according to claim 8, wherein the tail part is a thread produced from the pressed cotton or the hydrophilic cotton material.
